(19) United States
(12) Patent Application Publication
(10) Pub. No.: US 2020/0194115 A1
(43) Pub. Date: Jun. 18, 2020

(54) SYSTEM AND METHOD FOR INCORPORATING A SCANNER INTO A VEHICLE

(71) Applicant: Toyota Motor Engineering & Manufacturing North America, Inc., Plano, TX (US)

(72) Inventor: Chungchih Chou, Ann Arbor, MI (US)

(21) Appl. No.: 16/223,461

(22) Filed: Dec. 18, 2018

(51) Int. Cl. G16H 40/63; G16H 50/20; A61B 5/00; A61B 5/05; A61B 6/00

(52) U.S. Cl. CPC G16H 40/63; G16H 50/20; A61B 5/0035; A61B 6/4405; A61B 5/05; A61B 5/6893; A61B 5/0077

(57) ABSTRACT

Example systems and methods relate to placing a body scanners at entrances to vehicles such as buses or cars. The scans generated by the various body scanners can be used to generate a profile for each user that is continuously updated over time. The profile for each user can be used for a variety of purposes such as identifying positive or negative health trends (e.g., weight loss or gain), or authenticating the user. Furthermore, the scans can be used to determine if a user is sick (e.g., has a fever), and if so, select a seat in the vehicle for the user that is away from other passengers or to adjust the climate control system of the vehicle.

### TECHNICAL FIELD

The subject matter described herein relates, in general, to a system and method for incorporating a scanner into a vehicle, and in particular, to placing scanners at the entrances of autonomous and non-autonomous vehicles. Overtime, the scanners can be used to create a profile for a user that can be used to detect possible health issues of the user, authenticate the user, and recommend clothing items to the user based on the weather or a calendar event.

### BACKGROUND

Body scanners, such as millimeter wave scanners, are commonly used for security applications at airports. For example, a user passes through the millimeter wave scanner and a 3D model of the user is generated. The 3D model of the user can be examined by an agent to determine if the user is carrying any prohibited items such as weapons or explosives.

While such body scanners are limited to security applications, the body scanners (and other types of scanners) have the ability to perform a variety of health related tasks such as determining if the user has a fever, determining the body composition of the user (e.g., BMI, or bone density), and determining if the user has a limp or is injured. However, because of privacy concerns, or a security-focused mindset, currently airport body scanners do not provide any health related data to the users.

Furthermore, with respect to body scanners at airports, there is currently no way to compare a previously generated 3D model for a user with a more recently generated 3D model. As may be appreciated, if multiple generated 3D models for a user could be linked or associated with each other, they could be used to identify health trends for a user such as rapid weight loss or weight gain. However, even if the 3D models generated by airports for a user could be linked or associated, many users do not fly enough to draw any health related conclusions from the 3D models.

### SUMMARY

In one embodiment, example systems and methods relate to placing a body scanners at entrances to vehicles such as buses or cars. The body scanner may be integrated into the vehicle entrance or may be standalone scanner that the users pass through. The scans generated by the various body scanners can be used to generate a profile for each user that is continuously updated over time. The profile for each user can be used for a variety of purposes such as identifying positive or negative health trends (e.g., weight loss or gain), or authenticating the user. Furthermore, the scans can be used to determine if a user is sick (e.g., has a fever), and if so, select a seat in the vehicle for the user that is away from other passengers or to adjust the climate control system of the vehicle. Because users make use of cars or buses at a much greater frequency than airplanes, the scans collected by the body scanners placed at the vehicles and busses may provide more up-to-date data that can be used to quickly identify positive or negative health trends for the users.

In one embodiment, a system for collecting biometric data about a passenger of a vehicle is disclosed. The system includes one or more processors and a memory communicably coupled to the one or more processors. The memory stores a scanning module including instructions that when executed by the one or more processors cause the one or more processors to: perform a scan of a passenger of a vehicle; and based on the scan of the passenger of the vehicle, generate biometric data regarding the passenger of the vehicle. The memory further stores a profile module including instructions that when executed by the one or more processors cause the one or more processors to: determine a profile of the passenger of the vehicle, the profile including previously generated biometric data; and add the generated biometric data to the profile of the passenger of the vehicle. The memory further stores a health module including instructions that when executed by the one or more processors cause the one or more processors to: compare the generated biometric data with the previously generated biometric data; and determine a health condition of the passenger based on the comparison.

In one embodiment, a method for collecting biometric data about a passenger of a vehicle is disclosed. The method includes: performing a scan of a passenger of a vehicle; based on the scan, generating biometric data regarding the passenger of the vehicle; determining a profile of the passenger of the vehicle; and adding the generated biometric data to the profile of the passenger of the vehicle.

In one embodiment, a non-transitory computer-readable medium for performing a scan for a passenger of a vehicle is disclosed. The non-transitory computer-readable medium includes instructions that when executed by one or more processors cause the one or more processors to: perform a scan of a passenger of a vehicle; based on the scan of the passenger of the vehicle, generate biometric data regarding the passenger of the vehicle; determine a profile of the passenger of the vehicle, the profile including previously generated biometric data; add the generated biometric data to the profile of the passenger of the vehicle; compare the generated biometric data with the previously generated biometric data; determine a health condition of the passenger based on the comparison; alert one or both of the passenger or a driver of the vehicle about the health condition; authenticate the passenger based on the generated biometric data; and allow the passenger entry into the vehicle based on the authentication.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

FIG. 1 illustrates one embodiment of a vehicle within which systems and methods disclosed herein may be implemented.

FIG. 2 illustrates one embodiment of a biometric data system.

FIG. 3 illustrates a flowchart of a method that is associated with determining a health condition based on a scan of a passenger.

FIG. 4 illustrates a flowchart of a method that is associated with updating a profile based on a scan.

FIG. 5 illustrates a flowchart of a method that is associated with recommending clothing items based on a scan.

FIG. 6 illustrates an example vehicle and scanner.

### DETAILED DESCRIPTION

Systems, methods, and other embodiments associated with incorporating a scanner into vehicles are disclosed. As described previously, body scanners, such as millimeter wave scanners, are used at airports for security purposes. However, because of the focus on security at airports, there is currently no way for a user to track or get access to their history of body scans. Such a history could be used to identify positive and negative health trends for the user. Furthermore, such scanners may be too expensive for individual users to keep in their homes.

Accordingly, to solve the problems associated with scanners noted above, in an embodiment, scanners are placed at entrances to vehicles such as busses or autonomous cars. Each time a passenger enters a vehicle they first pass through the scanner, and a scan of the passenger is generated. The scan can be used to generate biometric data for the passenger that includes a variety of health related metrics such as height, weight, body temperature, and bone density. The biometric data may include a 3D model of the passenger's body. In the short term, the biometric data can be used for purposes such as instructing the passenger that they have a fever, and recommending a location in the vehicle for the passenger to sit to avoid infecting other passengers.

In the long term, the biometric data can be used to create a health profile for the passenger. The health profile can include a collection of biometric data that has been generated for the passenger each time the passenger entered a vehicle through a scanner. As may be appreciated, the collection of biometric data can be used to identify and diagnose possible long term health issues associated with the passenger. These issues may include rapid weight loss or gain, changes in bone density, changes in pulse or blood pressure, changes in muscle mass, etc. Any detected issues could be presented to the passenger in a report, for example.

The scanner and vehicle combinations described herein provide many advantages. First, because passengers may use a vehicle multiple times a month, week, or even days, and may pass through a scanner before each ride, their associated profile and collected biometric data can be used to create a very robust health profile for each passenger. This is in contrast with medical scans performed at hospitals or by doctors, which may not be frequent enough to identify certain health problems or trends. Second, by incorporating scanners into points-of-entry for autonomous vehicles such as busses, a large number of passengers may be able to reap the benefits of frequent body scans without the costs associated with doctor visits or purchasing a personal scanner.

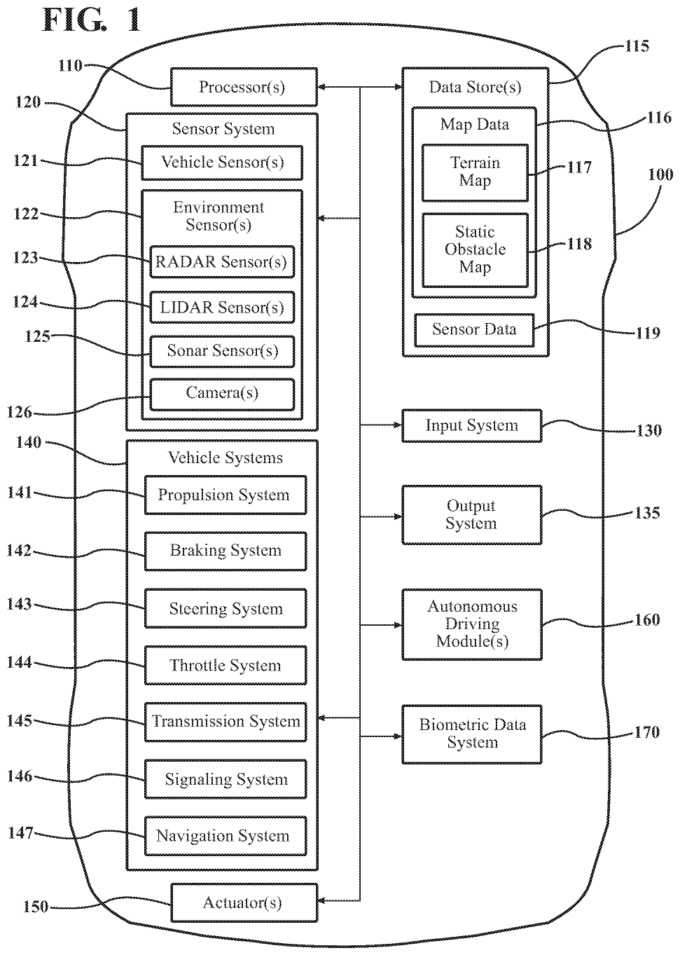

The vehicle 100 also includes various elements. It will be understood that in various embodiments it may not be necessary for the vehicle 100 to have all of the elements shown in FIG. 1. The vehicle 100 can have any combination of the various elements shown in FIG. 1. Further, the vehicle 100 can have additional elements to those shown in FIG. 1. In some arrangements, the vehicle 100 may be implemented without one or more of the elements shown in FIG. 1. While the various elements are shown as being located within the vehicle 100 in FIG. 1, it will be understood that one or more of these elements can be located external to the vehicle 100. Further, the elements shown may be physically separated by large distances.

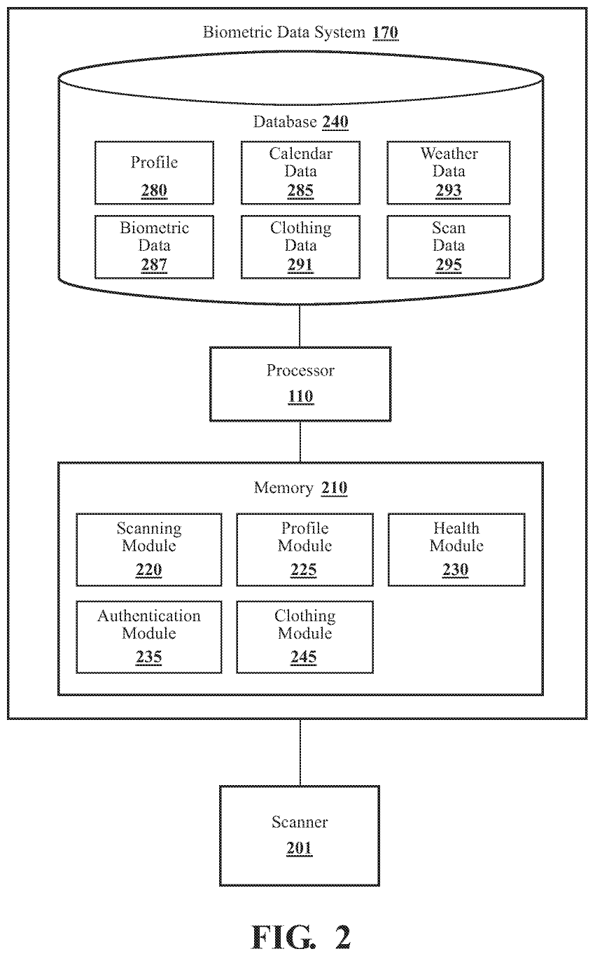

Some of the possible elements of the vehicle 100 are shown in FIG. 1 and will be described along with subsequent figures. However, a description of many of the elements in FIG. 1 will be provided after the discussion of FIGS. 2-6 for purposes of brevity of this description. Additionally, it will be appreciated that for simplicity and clarity of illustration, where appropriate, reference numerals have been repeated among the different figures to indicate corresponding or analogous elements. In addition, the discussion outlines numerous specific details to provide a thorough understanding of the embodiments described herein. Those of skill in the art, however, will understand that the embodiments described herein may be practiced using various combinations of these elements.

In either case, the vehicle 100 includes a biometric data system 170 that is implemented to perform methods and other functions as disclosed herein relating to incorporating a scanner into vehicle 100. The noted functions and methods will become more apparent with a further discussion of the figures.

With reference to FIG. 2, one embodiment of the biometric data system 170 of FIG. 1 is further illustrated. The biometric data system 170 is shown as including a processor 110 from the vehicle 100 of FIG. 1. Accordingly, the processor 110 may be a part of the biometric data system 170, the biometric data system 170 may include a separate processor from the processor 110 of the vehicle 100 or the biometric data system 170 may access the processor 110 through a data bus or another communication path. It should be appreciated, that while the biometric data system 170 is illustrated as being a single contained system, in various embodiments, the biometric data system 170 is a distributed system that is comprised of components that can be provided as a centralized server, a cloud-based service, and so on.

In one embodiment, the biometric data system 170 includes a memory 210 that stores a scanning module 220, a profile module 225, a health module 230, an authentication module 235, and a clothing module 245. The memory 210 is a random-access memory (RAM), read-only memory (ROM), a hard-disk drive, a flash memory, or other suitable memory for storing the modules 220, 225, 230, 235, and 245. The modules 220, 225, 230, 235, and 245 are, for example, computer-readable instructions that when executed by the processor 110 cause the processor 110 to perform the various functions disclosed herein. Moreover, as previously noted, in various embodiments, one or more aspects of the biometric data system 170 are implemented as cloud-based services, and so on. Thus, one or more modules of the biometric data system 170 may be located remotely from other components and may be implemented in a distributed manner.

Furthermore, in one embodiment, the biometric data system 170 includes the database 240. The database 240 is, in one embodiment, an electronic data structure stored in the memory 210 or another data store and that is configured with routines that can be executed by the processor 110 for analyzing stored data, providing stored data, organizing stored data, and so on. Thus, in one embodiment, the database 240 stores data used by the modules 220, 225, 230, 235, and 245 in executing various functions. In one embodiment, the database 240 includes a profile 280 along with, for example, other information that is used and/or generated by the modules 220, 225, 230, 235, and 245 such as calendar data 285, weather data 293, biometric data 287, clothing data 291, and scan data 295. Of course, in further embodiments, the various information may be stored within the memory 210 or another suitable location.

The scanning module 220 is configured to control a scanner 201 to scan a user or passenger of a vehicle 100 such as a bus or a car, for example. In some embodiments, the scanner 201 may be a full-body or biometric scanner such as a backscatter x-ray scanner and a millimeter wave scanner. Other types of scanners 201, or combinations of scanners 201, may be used such as ultrasound or MRI scanners. The scanning module 220 may interface with the scanner 201 using one or more wired or wireless technologies.

The scanner 201 may be a “full-body” scanner in that passengers may pass through the scanner 201 when entering (or alternatively exiting) the vehicle 100. For example, the scanner 201 may be placed in front of the entrance of a bus, and each passenger of the bus may pass through the scanner 201 when entering the bus. Depending on the embodiment, the scanner 201 may be a standalone scanner 201 that is not part of the vehicle 100, or the scanner 201 may be integrated into a door or entrance of the vehicle 100.

The scanning module 220 may be configured to scan a passenger of the vehicle before the passenger is permitted to enter the vehicle 100. For example, the scanning module 220 may control a door of the vehicle 100. In order to enter the vehicle 100, the passenger may first enter the scanner 201, where the passenger is scanned. After the scanning module 220 determines that the scan has been completed, the scanning module 220 may open the door of the vehicle 100 so that the passenger can enter. The process may be repeated by the scanning module 220 until all of the passengers of the vehicle 100 have been scanned.

In some embodiments, rather than scan the passenger using the scanner 201, the scanning module 220 may control one or more sensors of the sensor system 120 of the vehicle 100 to perform a scan of a passenger. These sensors may include one or more cameras, weight sensors, height sensors, etc. The scanning module 220 may use the sensors of the sensor system 120, instead of, or in addition to, the scanner 201, for example.

The scanning module 220 may be configured to receive or generate scan data 295 for a passenger scanned by the scanner 201 and/or sensors of the vehicle 100. In some embodiments, the scan data 295 may include a 3D rendering or model of the scanned passenger. Other information may be included in the scan data 295 depending on the type of scanner 201 and/or sensors used to generate the scan data 295 such as images and video (using various spectrums), a detected pulse, blood pressure, temperature, and weight of the passenger, ultrasound or MRI data, etc. Other types of data may be included in the scan data 295.

The profile module 225 may be configured to create, store, and maintain profiles 280 for passengers. The profile 280 for a passenger may uniquely identify the passenger using one or both of a name and an alphanumeric identifier. When a passenger is scanned, the profile module 225 may retrieve the profile 280 associated with the passenger, and if it exists, the profile module 225 may add some or all of the resulting scan data 295 to the profile 280. For example, the profile module 225 may add the 3D model of the passenger to the profile 280.

In some embodiments, when the passenger enters the scanner 201, the passenger may identify themselves to the profile module 225 using a card, dongle, fob, or some combination of user name and password. Once identified, the profile module 225 may retrieve the profile 280 associated with the passenger, and if none exists, may create and store a profile 280 for the passenger. As will be described further below, in some embodiments, the authentication module 235 may authenticate the passenger using scan data 295, and may instruct the profile module 225 which profile 280 corresponds to the passenger in the scanner 201, for example.

The health module 230 may be configured to extract/generate biometric data 287 from the scan data 295, and to add the biometric data 287 to the profile 280 associated with the passenger. Depending on the embodiment, the biometric data 287 may generally be any data or measurement that is associated with the health and/or well-being of the passenger such as body temperature, body shape, gait, posture (standing or walking), BMI, muscle-content, bone density, height, pulse, blood pressure, etc.

The health module 230 may be configured to determine one or more health conditions of the passenger based on the biometric data 287. These many include, but are not limited to, high/low blood pressure, fever, low bone density, injuries due to gait or posture, dehydration, etc. Other health conditions may be determined.

The health module 230 may be further configured to compare the biometric data 287 with previously generated biometric data 287 from the profile 280 associated with the passenger to determine additional health conditions. As may be appreciated, some health conditions, such as rapid weight gain or weight loss, may only be determined by comparing current biometric data 287 with previously generated biometric data 287. These conditions include loss of hair, muscle mass, bone density, etc.

The health module 230 may be further configured to alert the passenger of any determined health conditions. For example, the health module 230 may display the health condition to the passenger on a display associated with the vehicle 100. Alternatively or additionally, the health module 230 may send a notification to a mobile phone or other device associated with the passenger.

The health module 230 may be further configured to select a seat for the passenger in the vehicle 100 based on the health conditions. For example, the health module 230 may determine to have all passengers that show symptoms of illness (e.g., fever) sit in the same general area of the vehicle 100. In another example, the health module 225 may determine to have passengers that may have trouble walking sit in an area of the vehicle 100 that is close to the door. The seat or area assigned to the passenger may be presented to the passenger on the display associated with the vehicle 100. Alternatively or additionally, the health module 230 may send a notification to the mobile phone or other device associated with the passenger that includes the seating assignment.

The health module 230 may be further configured to adjust one or more vehicle 100 components based on the health conditions determined for the passenger. The adjustments may include adjusting the temperature of the vehicle 100, or adjusting the height of a seat in the vehicle 100 to better accommodate a sick or frail passenger. Other adjustments may be made.

The authentication module 235 may authenticate the passenger based on the scan data 295 and/or the biometric data 287 collected for the passenger. In some embodiments, when a passenger enters the scanner 201 and is scanned, the resulting scan data 295 and/or biometric data 287 is used to identify the profile 280 associated with the passenger. For example, the authentication module 235 may use the 3D scan of the passenger to locate a profile 280 that includes a matching, or partially matching, 3D scan. In another example, the authentication module 235 may locate the profile 285 using biometric data 287 such as eye color, gait, and the average or typical weight and height for the passenger as indicated in the profile 280.

The authentication module 235 may be configured to allow or deny a passenger access to a vehicle 100 based on the authentication. For example, if the authentication module 235 cannot find a matching profile 280 for the passenger, the authentication module 235 may deny the passenger entry into the vehicle 100. In another example, the authentication module 235 may maintain a list or file that includes passengers who are not permitted to ride in the vehicle 100. When the authentication module 235 finds a profile 280 for a passenger that is not permitted to ride the vehicle 100, the authentication module 235 may deny the passenger entry into the vehicle 100.

The clothing module 245 may be configured to determine clothing data 291 for a passenger based on the scan data 295 captured by the scanner 201 for a passenger. The clothing data 291 may identify one or more articles of clothing that the passenger was wearing when the passenger entered the scanner 201. The articles of clothing may include jackets, hats, pants, shirts, etc. The clothing data 291 may further identify accessories worn by the passenger such as glasses, jewelry, and an umbrella. Depending on the embodiment, the clothing data 291 may be determined by the clothing module 245 from the scan data 295. Alternatively, the clothing module 245 may determine the clothing data 291 from image data generated by one or more cameras or other sensors associated with the vehicle 100.

The clothing module 245 may be configured to detect one or more defects in the one or more articles of clothing worn by the passenger. The defects may include rips, tears, missing buttons, stains, etc. Other types of defect may be supported. The clothing module 245 may detect the defects in the articles of clothing using computer vision and the scan data 295. For example, the clothing module 245 may use a model trained to identify defects in clothing to detect the one or more defects. Other methods may be used. If the clothing module 245 detects a defect it may inform the passenger by sending the passenger a text message or email, for example.

The clothing module 245 may be further configured to recommend additional clothing items to the passenger based on the clothing data 291 generated for the passenger and one or both of calendar data 285 and weather data 293. With respect to calendar data 285, the clothing module 245 may retrieve calendar data 285 associated with the passenger. The calendar data 285 may include indications of upcoming events or meetings for the passenger. If the clothing module 245 determines that the passenger has an upcoming event that may require a certain clothing item (e.g., a business suit for an upcoming client meeting), the clothing module 245 may determine if such a clothing item is part of the clothing data 291. If not, the clothing module 245 may recommend the clothing item to the passenger. Depending on the embodiment, the clothing module 245 may include a list of calendar events and each event may be associated with a list of clothing items that are appropriate for the type of event.

With respect to weather data 293, the clothing module 245 may retrieve weather data 293 for a current location and/or a destination of the vehicle 100. The clothing module 245 may retrieve the weather data 293 from a weather syndication service, for example. Based on the weather data 293, the clothing module 245 may decide if the clothing items and accessories determined for the passenger as indicated in the clothing data 291 are appropriate for the weather indicated by the weather data 293. For example, if the weather data 293 indicates that it will be cold, the clothing module 245 may determine if the clothing data 291 indicates that the passenger is wearing a suitable coat. In another example, if the weather data 293 indicates that it will rain, the clothing module 245 may determine if the clothing data 291 indicates that the passenger has an umbrella or raincoat. If not, the clothing module 245 may recommend appropriate clothing items to the passenger based on the weather data 293. Depending on the embodiment, the clothing module 245 may include a list of weather conditions and each condition may be associated with a list of clothing items or accessories that are appropriate for the condition.

Note that while the scanner 201 and biometric data system 170 is described as being used in conjunction with a vehicle 100, it is not limited to such an application. For example, the scanner 201 and biometric system 170 may also be incorporated into a variety of entrances such as stadiums, fairgrounds, theaters, mall, office buildings, etc. The more entrances that incorporate the scanner 201 and biometric data system 170, the more accurate the generated profiles 280 may become for the users that opt-in or elect to have their health monitored by the biometric data system 170.

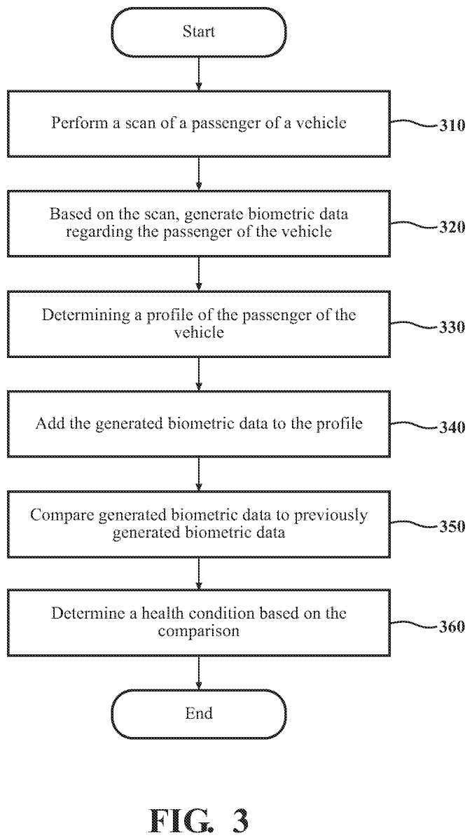

Additional aspects of scanning passengers will be discussed in relation to FIG. 3. FIG. 3 illustrates a flowchart of a method 300 that is associated with determining a health condition based on a scan of a passenger. The method 300 will be discussed from the perspective of the biometric data system 170 of FIGS. 1 and 2. While the method 300 is discussed in combination with the biometric data system 170, it should be appreciated that the method 300 is not limited to being implemented within the biometric data system 170 but is instead one example of a system that may implement the method 300.

At 310, the scanning module 220 performs a scan of a passenger of a vehicle. The vehicle 100 may be a car or a bus, for example. The scanning module 220 may perform the scan by instructing a scanner 201 to perform the scan of the passenger. The scanner 201 may be a full-body scanner such as a backscatter x-ray scanner, a millimeter wave scanner, or an MRI scanner. Other types of scanners 201 may be used. The scanner 201 may be placed in front of an entrance to the vehicle 100, so that the passenger may pass through the scanner 201 to gain entry into the vehicle 100. Alternatively, the scanner 201 may be incorporated into the entrance of the vehicle 100. Depending on the embodiment, rather than a full-body scanner 201, the scan of the passenger may be performed using one or more sensors associated with the vehicle 100 such as a camera 126. Other sensors may be used. The scanner 201 may generate scan data 295 based on the scan of the passenger.

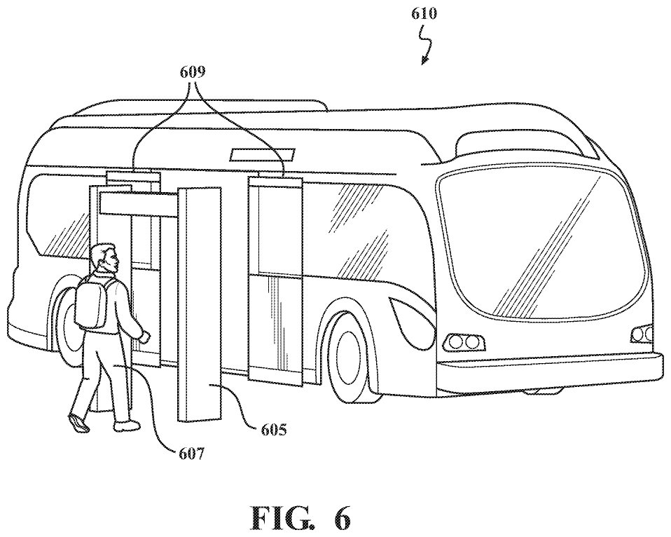

An example scanner 201 is illustrated in FIG. 6 as the scanner 605. The scanner 605 is located near doors 609 of a vehicle 610. As shown, the vehicle 610 is a bus, and a passenger 607 is walking through the scanner 605 in order to gain entry into the vehicle 610. After the passenger 607 passes through the scanner 605, scan data 295 is generated for the passenger 607, and the passenger 607 is permitted to enter the vehicle 610 through the doors 609.

Returning to FIG. 3, at 320, the health module 230 may generate biometric data 287 regarding the passenger of the vehicle 100. The health module 230 may generate the biometric data 287 based on the scan of the passenger performed by the scanner 201. More specifically, the biometric data 287 may be generated based on scan data 295 provided by the scanner 201. Depending on the embodiment, the biometric data 287 may include any data about the health or well-being of the passenger such as eye color, pulse, height, weight, BMI, gait, body temperature, bone density, blood pressure, etc. The biometric data 287 may further include a 3D model of the passenger. Other types of data may be included.

At 330, the profile module 225 may determine a profile 280 of the passenger of the vehicle 100. As described above, each passenger may have their own profile 280 that includes information about the passenger such as biometric data 287. Note that the profile 280 for a passenger may not be specific to a particular vehicle 100, but may be shared and updated by many different vehicles 100.

Depending on the embodiment, the profile module 225 may determine the profile 280 for a passenger based on the biometric data 287. For example, the authentication module 235 may use the biometric data 287 to determine a profile 280 that matches the biometric data 287 generated at 320. The authentication module 235 may then provide the matching profile 280 to the profile module 225. Alternatively, the profile 280 for a passenger may be determined by the profile module 225 using a card or dongle associated with the passenger, or by having the passenger login or otherwise identify themselves.

At 340, the health module 230 may add the generated biometric data 287 to the profile 280 associated with the passenger. As described above, the profile 280 for a passenger may include biometric data 287 collected from a variety of vehicles 100 and/or scanners 201. Because a passenger may use one or more vehicles 100 frequently, perhaps as part of a daily commute to work, the biometric data 287 contained in the profile 280 may be a robust and thorough representation of the health of the passenger. For example, the biometric data 287 may include an average or typical value for a variety of health metrics for the passenger such as average weight, average height, average blood pressure, average pulse, etc.

At 350, the health module 230 may compare the generated biometric data 287 with previously generated biometric data 287 from the profile 280. For example, the health module 230 may compare the 3D model of the passenger from the biometric data 287 generated at 320 with the 3D model of the passenger from the profile 280. In another example, the health module 230 may compare values such as average weight, average pulse, and average blood pressure with the corresponding more recent values from the biometric data generated at 320. Any method or technique for comparing values may be used.

At 360, the health module 230 may determine a health condition for the passenger based on the comparison. The health conditions may include, but are not limited to, weight gain, weight loss, high or low blood pressure, and decreasing muscle or bone density. Other health conditions may be supported.

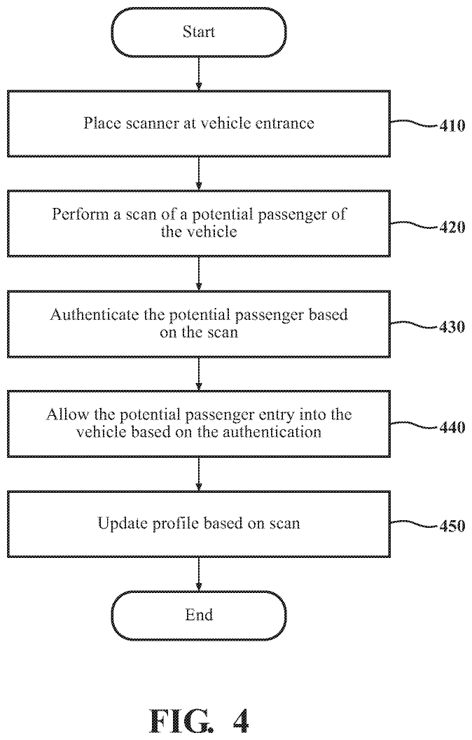

Additional aspects of scanning passengers will be discussed in relation to FIG. 4. FIG. 4 illustrates a flowchart of a method 400 that is associated with updating a profile based on a scan. The method 400 will be discussed from the perspective of the biometric data system 170 of FIGS. 1 and 2. While the method 400 is discussed in combination with the biometric data system 170, it should be appreciated that the method 400 is not limited to being implemented within the biometric data system 170 but is instead one example of a system that may implement the method 400.

At 410, a scanner 201 is placed at the entrance of a vehicle 100. The scanner 201 may be a full-body scanner such as a backscatter x-ray scanner, millimeter wave scanner, or an MRI scanner. Other types of scanners 201 may be used. The scanner 201 may be placed in front of an entrance to the vehicle 100, so that the passenger may pass through the scanner 201 to gain entry into the vehicle 100. Alternatively, the scanner 201 may be incorporated into the entrance of the vehicle 100. The scanner 201 may one of a plurality of scanners 201 that are placed at the entrances of a variety of different vehicles 100.

Note that the use of the scanner 201 and the biometric data system 170 are not limited to vehicles 100. For example, the scanners 201 may be placed at the entrance of a variety of places such as entrances to buildings such as stores, train stations, libraries offices, stadiums, restaurants, and other retail establishments.

At 420, the scanning module 220 performs a scan of a potential passenger of the vehicle 100. The vehicle 100 may be a car or a bus, for example. The scanning module 220 may perform the scan by instructing the scanner 201 to perform the scan of the potential passenger when the potential passenger enters the scanner 201. The scan of the potential passenger may result in the generation of biometric data 287 regarding the potential passenger. Depending on the embodiment, the potential passenger may not be permitted entry into the vehicle 100 until the scan is complete and the potential passenger has been authenticated.

At 430, the authentication module 235 authenticates the potential passenger based on the scan. The authentication module 235 may authenticate the potential passenger using the scan data 295 and/or the biometric data 287. For example, the authentication module 235 may search for a stored profile 280 for a passenger that has a similar 3D model as the potential passenger. Other biometric data 287 may be used to authenticate the potential passenger such as eye-color, average weight, average height, gait, average pule, or average blood pressure.

At 440, the authentication module 235 may allow the potential passenger to enter the vehicle 100 based on the authentication. For example, the authentication module 235 may send an instruction or signal to the vehicle 100 to open its entrance to allow the potential passenger to enter the vehicle 100.

At 450, the profile module 225 updates the profile 280 of the potential passenger. Depending on the embodiment, the profile module 225 may update the profile 280 of the potential passenger by adding some or all of the scan data 295 and/or biometric data 287 related to the scan performed at 420 to the profile 280 of the potential passenger. In addition, the profile module 225 may further update the profile 280 to reflect that the potential passenger has entered the vehicle 100. Such information may be used later for billing purposes, for example.

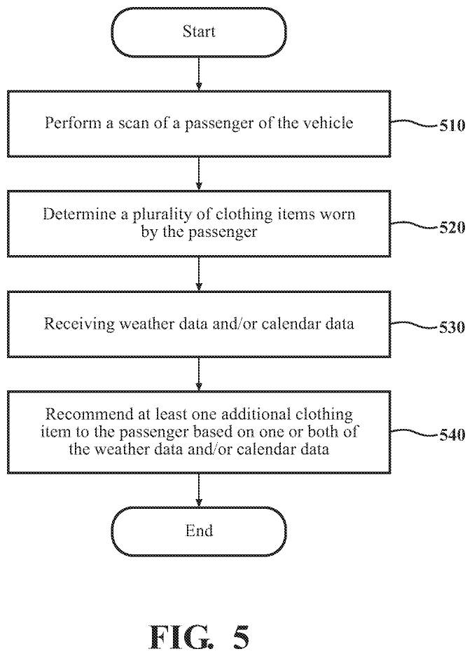

Additional aspects of scanning passengers will be discussed in relation to FIG. 5. FIG. 5 illustrates a flowchart of a method 500 that is associated with recommending clothing items based on a scan. The method 500 will be discussed from the perspective of the biometric data system 170 of FIGS. 1 and 2. While the method 500 is discussed in combination with the biometric data system 170, it should be appreciated that the method 500 is not limited to being implemented within the biometric data system 170 but is instead one example of a system that may implement the method 500.

At 510, the scanning module 220 performs a scan of a passenger of the vehicle 100. The vehicle 100 may be a car or a bus, for example. The scanning module 220 may perform the scan by instructing a scanner 201 to perform the scan of the passenger when the passenger enters the scanner 201. The scan of the passenger may result in the generation of scan data 295. Alternatively or additionally, the vehicle 100 may scan the passenger using one or more sensors such as a camera. Other types of sensors may be used.

At 520, the clothing module 245 may determine a plurality of clothing items worn by the passenger based on the scan. Depending on the embodiment, the clothing module 245 may determine the plurality of clothing items from the scan data 295. The plurality of clothing items may include jackets, shirts, dresses, etc. The plurality of clothing items may further include accessories such as glasses, hats, gloves, and umbrellas. Any method for determining clothing items from scan data 295 may be used such as object recognition or computer vision, for example.

At 530, the clothing module 245 receives one or both of weather data 293 or calendar data 285. The weather data 293 may indicate the current weather (e.g., temperature, chance of precipitation, humidity, or wind speed). The weather data 293 may be associated with a current location of the vehicle 100 or a future location of the vehicle 100. The clothing module 245 may request the weather data 293 from a weather syndication service, for example.

The calendar data 285 may be a calendar associated with the passenger of the vehicle 100. The calendar data 285 may include information such as meetings and other events that are scheduled for the passenger.

At 540, the clothing module 245 may recommend at least one additional clothing item to the passenger. The clothing module 245 may recommend at least one clothing item based on one or both of the weather data 293 and the calendar data 285. With respect to the weather data 293, the clothing module 245 may determine that the clothes or accessories being worn by the passenger may not be appropriate for the weather. For example, if the weather data 293 indicates that there will be rain, and the passenger does not have a raincoat or an umbrella, the clothing module 245 may recommend that the passenger get a raincoat or an umbrella. In another example, if the calendar data 285 indicates that the passenger has an upcoming business meeting, and the passenger is not wearing a suit or tie, the clothing module 245 may recommend that the passenger get a suit or tie.

FIG. 1 will now be discussed in full detail as an example environment within which the system and methods disclosed herein may operate. In some instances, the vehicle 100 is configured to switch selectively between an autonomous mode, one or more semi-autonomous operational modes, and/or a manual mode. Such switching can be implemented in a suitable manner, now known or later developed. “Manual mode” means that all of or a majority of the navigation and/or maneuvering of the vehicle is performed according to inputs received from a user (e.g., human driver). In one or more arrangements, the vehicle 100 can be a conventional vehicle that is configured to operate in only a manual mode.

In one or more embodiments, the vehicle 100 is an autonomous vehicle. As used herein, “autonomous vehicle” refers to a vehicle that operates in an autonomous mode. “Autonomous mode” refers to navigating and/or maneuvering the vehicle 100 along a travel route using one or more computing systems to control the vehicle 100 with minimal or no input from a human driver. In one or more embodiments, the vehicle 100 is highly automated or completely automated. In one embodiment, the vehicle 100 is configured with one or more semi-autonomous operational modes in which one or more computing systems perform a portion of the navigation and/or maneuvering of the vehicle along a travel route, and a vehicle operator (i.e., driver) provides inputs to the vehicle to perform a portion of the navigation and/or maneuvering of the vehicle 100 along a travel route.

The vehicle 100 can include one or more processors 110. In one or more arrangements, the processor(s) 110 can be a main processor of the vehicle 100. For instance, the processor(s) 110 can be an electronic control unit (ECU). The vehicle 100 can include one or more data stores 115 for storing one or more types of data. The data store 115 can include volatile and/or non-volatile memory. Examples of suitable data stores 115 include RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The data store 115 can be a component of the processor(s) 110, or the data store 115 can be operatively connected to the processor(s) 110 for use thereby. The term “operatively connected,” as used throughout this description, can include direct or indirect connections, including connections without direct physical contact.

In one or more arrangements, the one or more data stores 115 can include map data 116. The map data 116 can include maps of one or more geographic areas. In some instances, the map data 116 can include information or data on roads, traffic control devices, road markings, structures, features, and/or landmarks in the one or more geographic areas. The map data 116 can be in any suitable form. In some instances, the map data 116 can include aerial views of an area. In some instances, the map data 116 can include ground views of an area, including 360-degree ground views. The map data 116 can include measurements, dimensions, distances, and/or information for one or more items included in the map data 116 and/or relative to other items included in the map data 116. The map data 116 can include a digital map with information about road geometry. The map data 116 can be high quality and/or highly detailed.

In one or more arrangements, the map data 116 can include one or more terrain maps 117. The terrain map(s) 117 can include information about the ground, terrain, roads, surfaces, and/or other features of one or more geographic areas. The terrain map(s) 117 can include elevation data in the one or more geographic areas. The map data 116 can be high quality and/or highly detailed. The terrain map(s) 117 can define one or more ground surfaces, which can include paved roads, unpaved roads, land, and other things that define a ground surface.

In one or more arrangements, the map data 116 can include one or more static obstacle maps 118. The static obstacle map(s) 118 can include information about one or more static obstacles located within one or more geographic areas. A “static obstacle” is a physical object whose position does not change or substantially change over a period of time and/or whose size does not change or substantially change over a period of time. Examples of static obstacles include trees, buildings, curbs, fences, railings, medians, utility poles, statues, monuments, signs, benches, furniture, mailboxes, large rocks, hills. The static obstacles can be objects that extend above ground level. The one or more static obstacles included in the static obstacle map(s) 118 can have location data, size data, dimension data, material data, and/or other data associated with it. The static obstacle map(s) 118 can include measurements, dimensions, distances, and/or information for one or more static obstacles. The static obstacle map(s) 118 can be high quality and/or highly detailed. The static obstacle map(s) 118 can be updated to reflect changes within a mapped area.

The one or more data stores 115 can include sensor data 119. In this context, “sensor data” means any information about the sensors that the vehicle 100 is equipped with, including the capabilities and other information about such sensors. As will be explained below, the vehicle 100 can include the sensor system 120. The sensor data 119 can relate to one or more sensors of the sensor system 120. As an example, in one or more arrangements, the sensor data 119 can include information on one or more LIDAR sensors 124 of the sensor system 120.

In some instances, at least a portion of the map data 116 and/or the sensor data 119 can be located in one or more data stores 115 located onboard the vehicle 100. Alternatively, or in addition, at least a portion of the map data 116 and/or the sensor data 119 can be located in one or more data stores 115 that are located remotely from the vehicle 100.

As noted above, the vehicle 100 can include the sensor system 120. The sensor system 120 can include one or more sensors. “Sensor” means any device, component and/or system that can detect, and/or sense something. The one or more sensors can be configured to detect, and/or sense in real-time. As used herein, the term “real-time” means a level of processing responsiveness that a user or system senses as sufficiently immediate for a particular process or determination to be made, or that enables the processor to keep up with some external process.

In arrangements in which the sensor system 120 includes a plurality of sensors, the sensors can work independently from each other. Alternatively, two or more of the sensors can work in combination with each other. In such case, the two or more sensors can form a sensor network. The sensor system 120 and/or the one or more sensors can be operatively connected to the processor(s) 110, the data store(s) 115, and/or another element of the vehicle 100 (including any of the elements shown in FIG. 1). The sensor system 120 can acquire data of at least a portion of the external environment of the vehicle 100 (e.g., nearby vehicles).

The sensor system 120 can include any suitable type of sensor. Various examples of different types of sensors will be described herein. However, it will be understood that the embodiments are not limited to the particular sensors described. The sensor system 120 can include one or more vehicle sensors 121. The vehicle sensor(s) 121 can detect, determine, and/or sense information about the vehicle 100 itself. In one or more arrangements, the vehicle sensor(s) 121 can be configured to detect, and/or sense position and orientation changes of the vehicle 100, such as, for example, based on inertial acceleration. In one or more arrangements, the vehicle sensor(s) 121 can include one or more accelerometers, one or more gyroscopes, an inertial measurement unit (IMU), a dead-reckoning system, a global navigation satellite system (GNSS), a global positioning system (GPS), a navigation system 147, and/or other suitable sensors. The vehicle sensor(s) 121 can be configured to detect, and/or sense one or more characteristics of the vehicle 100. In one or more arrangements, the vehicle sensor(s) 121 can include a speedometer to determine a current speed of the vehicle 100.

Alternatively, or in addition, the sensor system 120 can include one or more environment sensors 122 configured to acquire, and/or sense driving environment data. “Driving environment data” includes data or information about the external environment in which an autonomous vehicle is located or one or more portions thereof. For example, the one or more environment sensors 122 can be configured to detect, quantify and/or sense obstacles in at least a portion of the external environment of the vehicle 100 and/or information/data about such obstacles. Such obstacles may be stationary objects and/or dynamic objects. The one or more environment sensors 122 can be configured to detect, measure, quantify and/or sense other things in the external environment of the vehicle 100, such as, for example, lane markers, signs, traffic lights, traffic signs, lane lines, crosswalks, curbs proximate the vehicle 100, off-road objects, etc.

Various examples of sensors of the sensor system 120 will be described herein. The example sensors may be part of the one or more environment sensors 122 and/or the one or more vehicle sensors 121. However, it will be understood that the embodiments are not limited to the particular sensors described.

As an example, in one or more arrangements, the sensor system 120 can include one or more radar sensors 123, one or more LIDAR sensors 124, one or more sonar sensors 125, and/or one or more cameras 126. In one or more arrangements, the one or more cameras 126 can be high dynamic range (HDR) cameras or infrared (IR) cameras.

The vehicle 100 can include an input system 130. An “input system” includes any device, component, system, element or arrangement or groups thereof that enable information/data to be entered into a machine. The input system 130 can receive an input from a vehicle passenger (e.g., a driver or a passenger). The vehicle 100 can include an output system 135. An “output system” includes any device, component, or arrangement or groups thereof that enable information/data to be presented to a vehicle passenger (e.g., a person, a vehicle passenger, etc.).

The vehicle 100 can include one or more vehicle systems 140. Various examples of the one or more vehicle systems 140 are shown in FIG. 1. However, the vehicle 100 can include more, fewer, or different vehicle systems. It should be appreciated that although particular vehicle systems are separately defined, each or any of the systems or portions thereof may be otherwise combined or segregated via hardware and/or software within the vehicle 100. The vehicle 100 can include a propulsion system 141, a braking system 142, a steering system 143, throttle system 144, a transmission system 145, a signaling system 146, and/or a navigation system 147. Each of these systems can include one or more devices, components, and/or a combination thereof, now known or later developed.

The navigation system 147 can include one or more devices, applications, and/or combinations thereof, now known or later developed, configured to determine the geographic location of the vehicle 100 and/or to determine a travel route for the vehicle 100. The navigation system 147 can include one or more mapping applications to determine a travel route for the vehicle 100. The navigation system 147 can include a global positioning system, a local positioning system or a geolocation system.

The processor(s) 110, the biometric data system 170, and/or the autonomous driving module(s) 160 can be operatively connected to communicate with the various vehicle systems 140 and/or individual components thereof. For example, returning to FIG. 1, the processor(s) 110 and/or the autonomous driving module(s) 160 can be in communication to send and/or receive information from the various vehicle systems 140 to control the movement, speed, maneuvering, heading, direction, etc. of the vehicle 100. The processor(s) 110, the biometric data system 170, and/or the autonomous driving module(s) 160 may control some or all of these vehicle systems 140 and, thus, may be partially or fully autonomous.

The processor(s) 110, the biometric data system 170, and/or the autonomous driving module(s) 160 can be operatively connected to communicate with the various vehicle systems 140 and/or individual components thereof. For example, returning to FIG. 1, the processor(s) 110, the biometric data system 170, and/or the autonomous driving module(s) 160 can be in communication to send and/or receive information from the various vehicle systems 140 to control the movement, speed, maneuvering, heading, direction, etc. of the vehicle 100. The processor(s) 110, the biometric data system 170, and/or the autonomous driving module(s) 160 may control some or all of these vehicle systems 140.

The processor(s) 110, the biometric data system 170, and/or the autonomous driving module(s) 160 may be operable to control the navigation and/or maneuvering of the vehicle 100 by controlling one or more of the vehicle systems 140 and/or components thereof. For instance, when operating in an autonomous mode, the processor(s) 110, the biometric data system 170, and/or the autonomous driving module(s) 160 can control the direction and/or speed of the vehicle 100. The processor(s) 110, the biometric data system 170, and/or the autonomous driving module(s) 160 can cause the vehicle 100 to accelerate (e.g., by increasing the supply of fuel provided to the engine), decelerate (e.g., by decreasing the supply of fuel to the engine and/or by applying brakes) and/or change direction (e.g., by turning the front two wheels). As used herein, “cause” or “causing” means to make, force, compel, direct, command, instruct, and/or enable an event or action to occur or at least be in a state where such event or action may occur, either in a direct or indirect manner.

The vehicle 100 can include one or more actuators 150. The actuators 150 can be any element or combination of elements operable to modify, adjust and/or alter one or more of the vehicle systems 140 or components thereof to responsive to receiving signals or other inputs from the processor(s) 110 and/or the autonomous driving module(s) 160. Any suitable actuator can be used. For instance, the one or more actuators 150 can include motors, pneumatic actuators, hydraulic pistons, relays, solenoids, and/or piezoelectric actuators, just to name a few possibilities.

The vehicle 100 can include one or more modules, at least some of which are described herein. The modules can be implemented as computer-readable program code that, when executed by a processor 110, implement one or more of the various processes described herein. One or more of the modules can be a component of the processor(s) 110, or one or more of the modules can be executed on and/or distributed among other processing systems to which the processor(s) 110 is operatively connected. The modules can include instructions (e.g., program logic) executable by one or more processor(s) 110. Alternatively, or in addition, one or more data store 115 may contain such instructions.

In one or more arrangements, one or more of the modules described herein can include artificial or computational intelligence elements, e.g., neural network, fuzzy logic or other machine learning algorithms. Further, in one or more arrangements, one or more of the modules can be distributed among a plurality of the modules described herein. In one or more arrangements, two or more of the modules described herein can be combined into a single module.

The vehicle 100 can include one or more autonomous driving modules 160. The autonomous driving module(s) 160 can be configured to receive data from the sensor system 120 and/or any other type of system capable of capturing information relating to the vehicle 100 and/or the external environment of the vehicle 100. In one or more arrangements, the autonomous driving module(s) 160 can use such data to generate one or more driving scene models. The autonomous driving module(s) 160 can determine position and velocity of the vehicle 100. The autonomous driving module(s) 160 can determine the location of obstacles, obstacles, or other environmental features including traffic signs, trees, shrubs, neighboring vehicles, pedestrians, etc.

The autonomous driving module(s) 160 can be configured to receive, and/or determine location information for obstacles within the external environment of the vehicle 100 for use by the processor(s) 110, and/or one or more of the modules described herein to estimate position and orientation of the vehicle 100, vehicle position in global coordinates based on signals from a plurality of satellites, or any other data and/or signals that could be used to determine the current state of the vehicle 100 or determine the position of the vehicle 100 with respect to its environment for use in either creating a map or determining the position of the vehicle 100 in respect to map data.

The autonomous driving module(s) 160 either independently or in combination with the biometric data system 170 can be configured to determine travel path(s), current autonomous driving maneuvers for the vehicle 100, future autonomous driving maneuvers and/or modifications to current autonomous driving maneuvers based on data acquired by the sensor system 120, driving scene models, and/or data from any other suitable source such as determinations from the sensor data 250. “Driving maneuver” means one or more actions that affect the movement of a vehicle. Examples of driving maneuvers include: accelerating, decelerating, braking, turning, moving in a lateral direction of the vehicle 100, changing travel lanes, merging into a travel lane, and/or reversing, just to name a few possibilities. The autonomous driving module(s) 160 can be configured can be configured to implement determined driving maneuvers. The autonomous driving module(s) 160 can cause, directly or indirectly, such autonomous driving maneuvers to be implemented. As used herein, “cause” or “causing” means to make, command, instruct, and/or enable an event or action to occur or at least be in a state where such event or action may occur, either in a direct or indirect manner. The autonomous driving module(s) 160 can be configured to execute various vehicle functions and/or to transmit data to, receive data from, interact with, and/or control the vehicle 100 or one or more systems thereof (e.g., one or more of vehicle systems 140).

Detailed embodiments are disclosed herein. However, it is to be understood that the disclosed embodiments are intended only as examples. Therefore, specific structural and functional details disclosed herein are not to be interpreted as limiting, but merely as a basis for the claims and as a representative basis for teaching one skilled in the art to variously employ the aspects herein in virtually any appropriately detailed structure. Further, the terms and phrases used herein are not intended to be limiting but rather to provide an understandable description of possible implementations. Various embodiments are shown in FIGS. 1-6, but the embodiments are not limited to the illustrated structure or application.

The flowcharts and block diagrams in the figures illustrate the architecture, functionality, and operation of possible implementations of systems, methods, and computer program products according to various embodiments. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suited. A typical combination of hardware and software can be a processing system with computer-usable program code that, when being loaded and executed, controls the processing system such that it carries out the methods described herein. The systems, components and/or processes also can be embedded in a computer-readable storage, such as a computer program product or other data programs storage device, readable by a machine, tangibly embodying a program of instructions executable by the machine to perform methods and processes described herein. These elements also can be embedded in an application product which comprises all the features enabling the implementation of the methods described herein and, which when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. The phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: a portable computer diskette, a hard disk drive (HDD), a solid-state drive (SSD), a read-only memory (ROM), an erasable programmable read-only memory (EPROM or Flash memory), a portable compact disc read-only memory (CD-ROM), a digital versatile disc (DVD), an optical storage device, a magnetic storage device, or any suitable combination of the foregoing. In the context of this document, a computer-readable storage medium may be any tangible medium that can contain, or store a program for use by or in connection with an instruction execution system, apparatus, or device.

Generally, modules as used herein include routines, programs, objects, components, data structures, and so on that perform particular tasks or implement particular data types. In further aspects, a memory generally stores the noted modules. The memory associated with a module may be a buffer or cache embedded within a processor, a RAM, a ROM, a flash memory, or another suitable electronic storage medium. In still further aspects, a module as envisioned by the present disclosure is implemented as an application-specific integrated circuit (ASIC), a hardware component of a system on a chip (SoC), as a programmable logic array (PLA), or as another suitable hardware component that is embedded with a defined configuration set (e.g., instructions) for performing the disclosed functions.

Program code embodied on a computer-readable medium may be transmitted using any appropriate medium, including but not limited to wireless, wireline, optical fiber, cable, RF, etc., or any suitable combination of the foregoing. Computer program code for carrying out operations for aspects of the present arrangements may be written in any combination of one or more programming languages, including an object-oriented programming language such as Java™ Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The phrase “at least one of . . . and . . . ” as used herein refers to and encompasses any and all possible combinations of one or more of the associated listed items. As an example, the phrase “at least one of A, B, and C” includes A only, B only, C only, or any combination thereof (e.g., AB, AC, BC or ABC).

Aspects herein can be embodied in other forms without departing from the spirit or essential attributes thereof. Accordingly, reference should be made to the following claims, rather than to the foregoing specification, as indicating the scope hereof.

What is claimed is:

1. A system for collecting biometric data about a passenger of a vehicle, the system comprising:
one or more processors;
a memory communicably coupled to the one or more processors and storing:
a scanning module including instructions that when executed by the one or more processors cause the one or more processors to:
perform a scan of a passenger of a vehicle; and
based on the scan of the passenger of the vehicle, generate biometric data regarding the passenger of the vehicle;
a profile module including instructions that when executed by the one or more processors cause the one or more processors to:
determine a profile of the passenger of the vehicle, the profile including previously generated biometric data; and
add the generated biometric data to the profile of the passenger of the vehicle; and
a health module including instructions that when executed by the one or more processors cause the one or more processors to:
compare the generated biometric data with the previously generated biometric data; and
determine a health condition of the passenger based on the comparison.

2. The system of claim 1, wherein the health module further includes instructions that when executed by the one or more processors cause the one or more processors to:
alert one or both of the passenger or a driver of the vehicle about the health condition.

3. The system of claim 1, further comprising an authentication module including instructions that when executed by the one or more processors cause the one or more processors to:
authenticate the passenger based on the generated biometric data; and
allow the passenger entry into the vehicle based on the authentication.

4. The system of claim 1, wherein the health condition comprises one or more of weight loss or weight gain.

5. The system of claim 1, further comprising a clothing module including instructions that when executed by the one or more processors cause the one or more processors to:
determine a plurality of clothing items worn by the passenger based on the scan;
receive weather data; and
based at least in part on the determined plurality of clothing items and the weather data, recommend at least one additional clothing item to the passenger, wherein the at least one additional clothing item is not part of the determined plurality of clothing items.

6. The system of claim 1, further comprising a clothing module including instructions that when executed by the one or more processors cause the one or more processors to:
determine a plurality of clothing items worn by the passenger based on the scan;
receiving calendar data associated with the passenger; and
based at least in part on the determined plurality of clothing items and the calendar data, recommend at least one additional clothing item to the passenger, wherein the at least one additional clothing item is not part of the determined plurality of clothing items.

7. A method for collecting biometric data about a passenger of a vehicle, the method comprising:
performing a scan of a passenger of a vehicle;
based on the scan, generating biometric data regarding the passenger of the vehicle;
determining a profile of the passenger of the vehicle; and
adding the generated biometric data to the profile of the passenger of the vehicle.

8. The method of claim 7, wherein performing the scan comprises performing the scan using one or more of a camera, a backscatter x-ray scanner, and a millimeter wave scanner.

9. The method of claim 7, further comprising:
determining a health condition of the passenger based on the generated biometric data; and
alerting the passenger of the health condition.

10. The method of claim 9, further comprising:
alerting a driver of the vehicle of the health condition.

11. The method of claim 9, wherein the health condition comprising one or more of high blood pressure, a high pulse rate, or a fever.

12. The method of claim 7, wherein the profile of the passenger comprises previously generated biometric data, and further comprising:
comparing the generated biometric data with the previously generated biometric data; and
determining a health condition of the passenger based on the comparison.

13. The method of claim 12, wherein the health condition comprises one or more of weight loss or weight gain.

14. The method of claim 7, further comprising:
determining a problem with one or more items of clothing associated with the passenger based on the scan.

15. The method of claim 7, wherein performing the scan of the passenger of the vehicle comprises performing the scan before the passenger enters the vehicle.

16. The method of claim 7, further comprising:
determining a plurality of clothing items worn by the passenger based on the scan;
receiving weather data; and
based at least in part on the determined plurality of clothing items and the weather data, recommending at least one additional clothing item to the passenger, wherein the at least one additional clothing item is not part of the determined plurality of clothing items.

17. The method of claim 7, further comprising:
determining a plurality of clothing items worn by the passenger based on the scan;
receiving calendar data associated with the passenger; and
based at least in part on the determined plurality of clothing items and the calendar data, recommending at least one additional clothing item to the passenger, wherein the at least one additional clothing item is not part of the determined plurality of clothing items.

18. The method of claim 7, wherein the vehicle comprises an autonomous vehicle.

19. The method of claim 7, further comprising:
authenticating the passenger based on the generated biometric data; and
allowing the passenger entry into the vehicle based on the authentication.

20. A non-transitory computer-readable medium for performing a scan for a passenger of a vehicle and including instructions that when executed by one or more processors cause the one or more processors to:
perform a scan of a passenger of a vehicle;
based on the scan of the passenger of the vehicle, generate biometric data regarding the passenger of the vehicle;
determine a profile of the passenger of the vehicle, the profile including previously generated biometric data;
add the generated biometric data to the profile of the passenger of the vehicle;
compare the generated biometric data with the previously generated biometric data;
determine a health condition of the passenger based on the comparison;
alert one or both of the passenger or a driver of the vehicle about the health condition;
authenticate the passenger based on the generated biometric data; and
allow the passenger entry into the vehicle based on the authentication.
